(19) United States
(12) Patent Application Publication
(10) Pub. No.: US 2023/0389846 A1
(43) Pub. Date: Dec. 7, 2023

(54) WIRELESS MULTI-ELECTRODE POTENTIAL DETECTION DEVICE USING VACUUM SUCTION AND METHOD FOR USE THEREOF

(71) Applicant: Niyant Vora, Bloomington, IL (US)

(72) Inventor: Niyant Vora, Bloomington, IL (US)

(21) Appl. No.: 17/831,118

(22) Filed: Jun. 2, 2022

(51) Int. Cl. A61B 5/252

(52) U.S. Cl. CPC A61B 5/252

(57) ABSTRACT

A biopotential detection device configured to be held by vacuum to a skin area. An embodiment includes a housing having an end wall and a skirt extending away from the top portion in a first direction. The skirt includes a bottom edge disposed away from the end wall and configured to engage the skin area. A void defined by the skirt, the end wall and the skin surface forms a vacuum chamber. The device also includes a cover disposed adjacent to the end wall. The device also includes a vacuum port extending through the cover and the top portion and into the vacuum chamber. The device also includes one or more electrodes disposed on the bottom edge of the skirt.

### TECHNICAL FIELD

The present application relates to a wireless multi-electrode potential detection device using vacuum suction and a method for use thereof. More particularly, the present disclosure relates to systems and methods for multi-electrode biopotential detection and noise elimination devices.

### BACKGROUND

Unless otherwise indicated herein, the materials, systems and/or processes described in this section are not prior art to the claims in this application and are not admitted to be prior art by inclusion in this section.

Generally, a variety of physiological parameters or other information about the health state of a person can be detected by electrodes. Electrodes are conductors though which an electrical current can flow. Electrodes can non-invasively be attached to a body part of a patient and measure biopotentials in the body. The biopotentials are measured between two electrodes on the patient. The information can then be transferred from the body part to a machine that measures and records the information. In order for electrodes to properly measure biopotentials, they must be securely attached, such as to the skin of a patient. Electrodes can typically be non-invasively attached through an adhesive patch, on a harness fitting snuggly to a patient, or with conductive adhesive gel.

### SUMMARY

This summary is illustrative only and is not intended to be in any way limiting. Other aspects, inventive features, and advantages of the devices or processes described herein will become apparent in the detailed description set forth herein, taken in conjunction with the accompanying FIGS., wherein like reference numerals refer to like elements.

Some embodiments of the present disclosure provide a biopotential detection device held by a vacuum to a skin area. The device includes a housing having an end wall, and a skirt extending away from the top portion in a first direction. The skirt includes a bottom edge disposed away from the end wall and configured to engage a skin area. A void is at least partially defined by the skirt, the end wall, and the skin area. The void forms a vacuum chamber therein. The device also includes a cover disposed adjacent to the end wall, opposite the skirt. The device also includes a vacuum port extending through the cover and the end wall and into the vacuum chamber. The device also includes one or more electrodes disposed on the bottom edge of the skirt.

Some embodiments of the present disclosure provide a housing having an end wall, a lip extending around a circumference of the top portion and away from the top portion in a first direction, a skirt extending away from the end wall in a second direction, oblique from the first direction, and a raised central portion having a housing opening extending there through. The skirt at least partially defines a vacuum chamber. The housing opening defines a fluid pathway defined at least in part by an exterior of the housing and the vacuum chamber.

Some embodiments of the present disclosure provide a method for positioning a biopotential detection device on a skin area. The method includes positioning a biopotential detection device on the skin area. The biopotential detection device comprises a housing having a bottom edge that contacts the skin area and a vacuum chamber defined at least in part by the skin area and the housing. The method also includes securing the housing to the skin area such that the at least two electrodes are in contact with the skin area. The method also includes suctioning air out of the vacuum chamber to as to create a vacuum seal between the housing and the skin area to secure the electrodes on the skin area.

This summary is illustrative only and is not intended to be in any way limiting. Other aspects, inventive features, and advantages of the devices or processes described herein will become apparent in the detailed description set forth herein, taken in conjunction with the accompanying FIGS., wherein like reference numerals refer to like elements. Additionally, one or more features of an aspect of the disclosure may be combined with one or more features of a different aspect of the disclosure.

### BRIEF DESCRIPTION OF THE DRAWINGS

FIG. 1 is a perspective view of a biopotential detection device, according to an example embodiment.

FIG. 2 is an exploded view of the biopotential detection device of FIG. 1.

FIG. 3 is a front view of the biopotential detection device of FIG. 1.

FIG. 4 is a top view of the biopotential detection device of FIG. 1.

FIG. 5 is a cross-sectional view of the biopotential detection device of FIG. 1.

FIG. 6 is a cross-sectional view of a body of the biopotential detection device of FIG. 1.

FIG. 7 is a side view showing aspects of the biopotential detection device of FIG. 1

FIG. 8 is a cross-sectional view showing aspects of the biopotential detection device of FIG. 1.

FIG. 9 is a flow diagram of a method of operating the biopotential detection device 100 of FIG. 1.

These and other features, together with the organization and manner of operation thereof, will become apparent from the following detailed description when taken in conjunction with the accompanying drawings.

### DETAILED DESCRIPTION

Following below are more detailed descriptions of various concepts related to, and implementations of methods, apparatuses, and systems for a wireless multi-electrode potential detection device using vacuum suction. The various concepts introduced herein may be implemented in any number of ways, as the concepts described are not limited to any particular manner of implementation. Examples of specific implementations and applications are provided primarily for illustrative purposes.

Referring to the Figures generally, the various embodiments disclosed herein relate to systems, apparatuses, and methods for a biopotential detection device for detecting one or more biopotential signals. The biopotential detection device includes a plurality (e.g., at least two) electrodes configured to detect a biopotential signal (e.g., voltage). Accordingly, the biopotential detection device is positionable on a skin surface. The biopotential detection device may be secured to the skin surface by a vacuum seal. The biopotential detection device is advantageously wireless in that the biopotential detection device may transmit detected biopotential signals wirelessly to a remote computing device. These and other features and benefits are described more fully herein below.

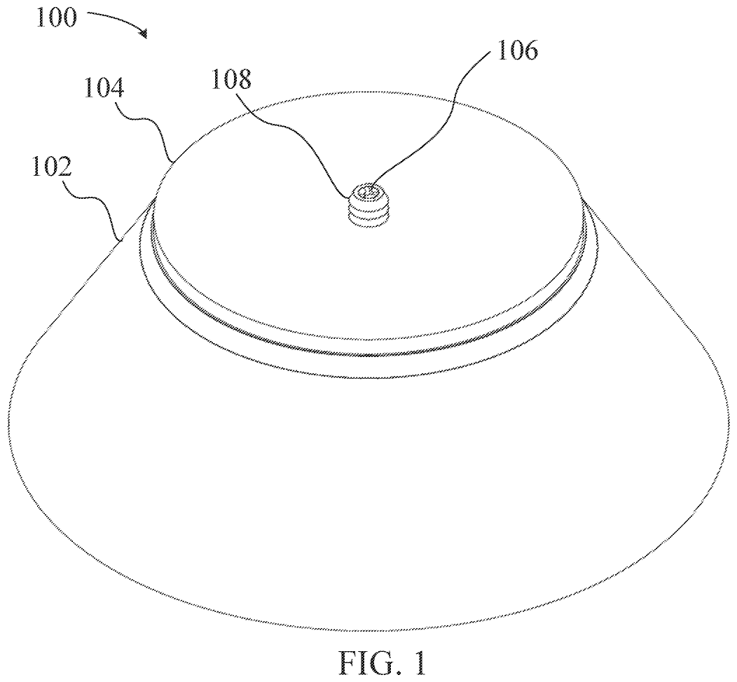

Referring now to FIG. 1, a biopotential detection device 100 is shown, according to an example embodiment. The biopotential detection device 100 includes a housing 102, a cover 104, and a hose fitting 108. The hose fitting at least partially defining a vacuum port 106. The housing 102 may provide structural support for the other components of the biopotential detection device 100. The housing 102 and/or the cover 104 may serve as the main article of contact for a user to manipulate and position the biopotential detection device 100 for use.

The hose fitting 108 is structured to sealably couple to an inner diameter of a hose (not shown). For example, the hose fitting 108 includes one or more barbs for sealably coupling to a hose. As briefly described above, the hose fitting 108 at least partially defines a vacuum port 106. When a hose is coupled to the hose fitting 108, the hose is in fluid communication with the vacuum port 106. The vacuum port 106 extends through the cover 104 and the housing 102 and defines a fluid pathway therein such that an outside of the biopotential detection device 100 is in fluid communication with an inside of the biopotential detection device 100 via the vacuum port 106.

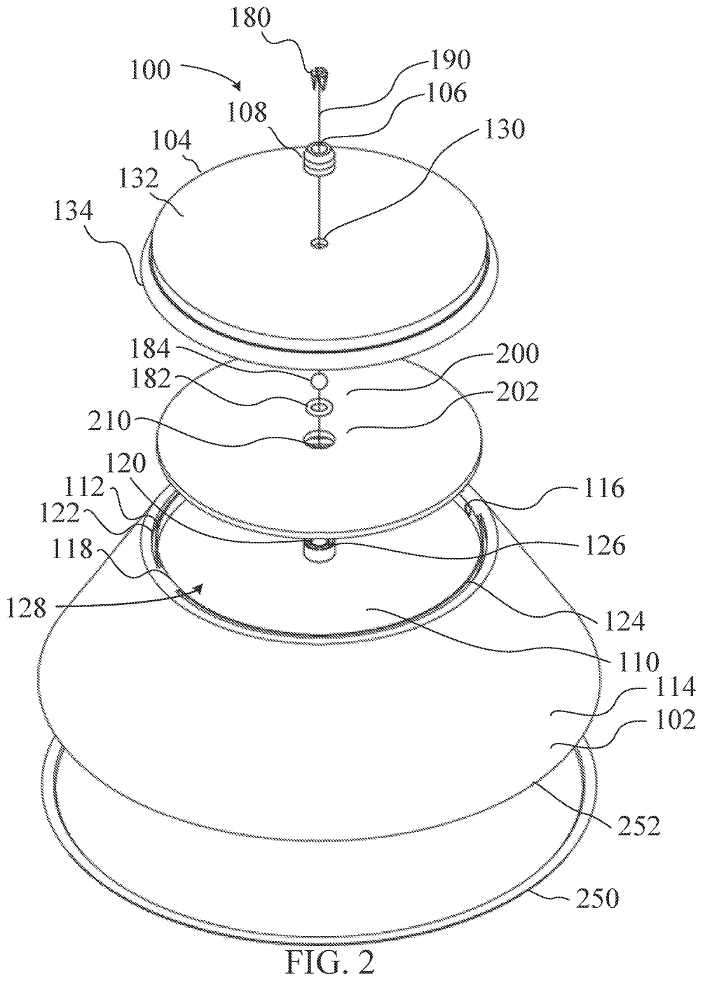

FIG. 2 is an exploded view of the biopotential detection device 100, according to an embodiment. In the illustrated embodiment, the biopotential detection device 100 includes the housing 102, the cover 104, and the hose fitting 108 of FIG. 1. The biopotential detection device 100 also includes control circuitry shown as a control circuit 200. The biopotential detection device 100 also includes a pronged member 180, a gasket 182, and a ball gasket 184. In some embodiments and as shown in FIG. 2, the biopotential detection device 100 optionally includes an adhesive ring 250. A central axis 190 is defined through a center of the biopotential detection device 100. Accordingly a radial direction is defined as a direction perpendicular to the central axis 190, and a circumferential direction is defined as a direction around the central axis 190 (e.g., in a substantially circular shape).

In one embodiment, the housing 102 includes an end wall 110, a circumferential lip 112 that extends around a circumference of the end wall 110 in a first direction, and a skirt 114 that extends away from the end wall in a second direction, oblique to the first direction. The housing 102 may also include a first channel 116 and a second channel 118. In one embodiment, the skirt 114 may comprise multiple components, such as an inner skirt and an outer skirt that fits on the inner skirt, with the first channel 116 and the second channel 118 defined between the inner skirt and outer skirt. For example, the first channel 116 may be formed by a groove in one of the inner or outer skirt that is sealed off along its length when the inner and outer skirt are engaged to form the skirt 114. The first channel 116 extends through the circumferential lip 112 and the skirt 114 at a first radial side, and the second channel 118 extends through the circumferential lip 112 and the skirt 114 at a second radial side, diametrically opposite the first radial side. The end wall 110 includes a raised central portion 126. A housing opening 120 is formed through the raised central portion 126. The housing opening 120 at least partially defines the vacuum port 106 such that the vacuum port extends through the raised central portion 126 and the end wall 110. The housing opening 120 is substantially aligned with a central axis 190. The circumferential lip 112 may define a first circumferential channel 122 and a second circumferential channel 124. The first circumferential channel 122 and the second circumferential channel 124 may be diametrically opposed on a third radial side and a fourth radial side, respectively. For example, the first circumferential channel 122 extends at least partially between the first channel 116 to the second channel 118 on the third radial side and the second circumferential channel extends at least partially between the first channel 116 to the second channel 118 on the fourth radial side.

The cover 104 includes a top surface 132 and an angled surface 134 that extends away from the top surface 132 in an oblique direction (e.g., the second direction) such that the angled surface 134 is substantially aligned with and/or parallel to the skirt 114, when the biopotential detection device 100 is assembled. The cover 104 includes a cover opening 130 that at least partially defines the vacuum port 106 such that the vacuum port 106 extends through the top surface 132 of the cover 104. The cover opening 130 is substantially aligned with the central axis 190. The hose fitting 108 may be coupled to the top surface 132 and/or fitted into the cover opening 130.

The control circuit 200 includes a controller 202 for controlling one or more biopotential sensing devices such as an electrode. The control circuit 200 may also include one or more communication interfaces for transmitting detected data to a remote device. The one or more communication interfaces may be communicatively coupled to the controller 202 or embodied on the controller 202. The one or more communication interfaces may include any combination of hardware and/or software for transmitting data over one or more of Bluetooth, Wi-Fi, NFC, etc. The control circuit 200 defines a circuit opening 210 that is substantial aligned with the raised central portion 126 such that the raised central portion 126 extends through the circuit opening 210. When the biopotential detection device 100 is assembled, the control circuit 200 is positioned in a cavity 128 defined, at least partially, by the end wall 110, the circumferential lip 112, the raised central portion 126, and the cover 104.

The pronged member 180 is positioned within the vacuum port 106 and proximal the hose fitting 108. In some embodiments, the pronged member 180 is fitted within the hose fitting 108. The pronged member 180 includes one or more prongs for temporarily retaining the ball gasket 184 when in operation. As shown, the pronged member includes three prongs.

The gasket 182 is a circular gasket (e.g., O-ring), and the ball gasket 184 is a spherical gasket. In operation, the gasket 182 and the ball gasket 184 form a fluid-tight seal in the vacuum port 106 such that a fluid cannot pass there through. When a fluid is being drawn out of the biopotential detection device 100 (e.g., by a vacuum) the ball gasket 184 is retained by the pronged member 180 to allow a fluid to be evacuated from the inside of the biopotential detection device 100. When a suitable amount of fluid has been removed from the biopotential detection device 100, the ball gasket 184 is returned to the gasket 182 and the seal is reformed preventing the fluid from re-entering the inside of the biopotential detection device 100.

As shown in FIG. 2, the housing opening 120, cover opening 130, the circuit opening 210, and the hose fitting 108 collectively define the vacuum port 106. The vacuum port 106, gasket 182, ball gasket 184, hose fitting 108, and pronged member 180 may be aligned along the same central axis 190 through the biopotential detection device 100.

When the biopotential detection device 100 is positioned on area surface, such as an area of skin, a vacuum seal may be formed between the housing 102 and the skin area and secures the biopotential detection device 100 to the skin area. Accordingly, the biopotential detection device 100 may advantageously include an adhesive ring 250 is a ring that extends around a bottom edge 252 of the skirt 114. The adhesive ring 250 defines a contact surface between the housing 102 and a surface such as skin. The adhesive ring 250 may function as a gasket between the skin and the biopotential detection device 100. In some embodiments, the adhesive ring 250 includes an adhesive or gel applied thereon. Various types of adhesives and gels may be used to secure the housing 102 to the skin area including acrylate, isooctyl acrylate, methyl-acrylate, or other suitable compound. In some embodiments, the adhesive or gel may be disposed in segments on a portion of the bottom edge 252 of the skirt 114. In other embodiments, the adhesive ring 250 is a grip surface made of a suitable material, such as reusable medical grade silicon or silane-derived materials. In some embodiments, the adhesive ring 250 is made of the same material as the skirt 114.

In some embodiments, the bottom edge 252 optionally defines a sealing surface. In embodiments where the bottom edge 252 includes the adhesive ring 250, the sealing surface may be defined on one or more sides (e.g., radially outside, radially inside, and/or circumferentially between segments) of the adhesive ring 250. In some embodiments, the sealing surface may be made of the same material as the bottom edge 252. In other embodiments, the sealing surface is made from a nitrile rubber, silicone, or other suitable material.

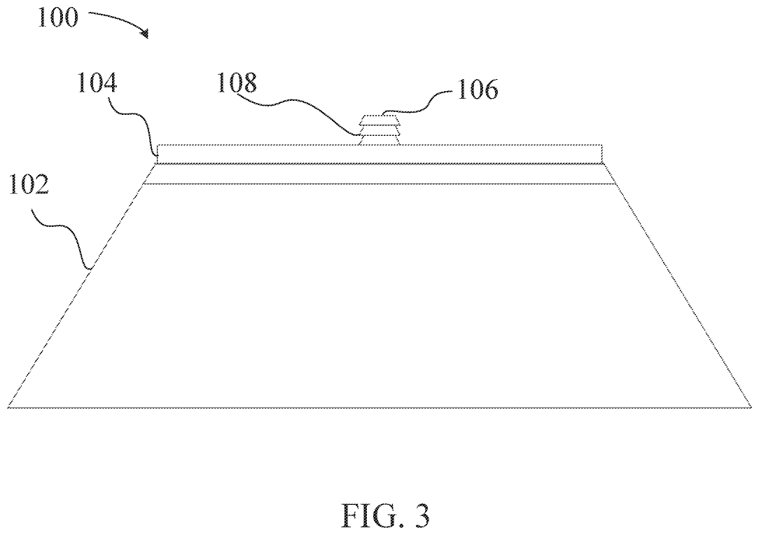

FIG. 3 is a front view of the biopotential detection device 100. In some embodiments, the housing 102 may take the shape of a cup, such as a suction cup. In some embodiments, the biopotential detection device 100 is substantially frustoconical in shape. In an example embodiment, the biopotential detection device 100 the skirt 114 defines an inside portion of the biopotential detection device 100 having an open bottom.

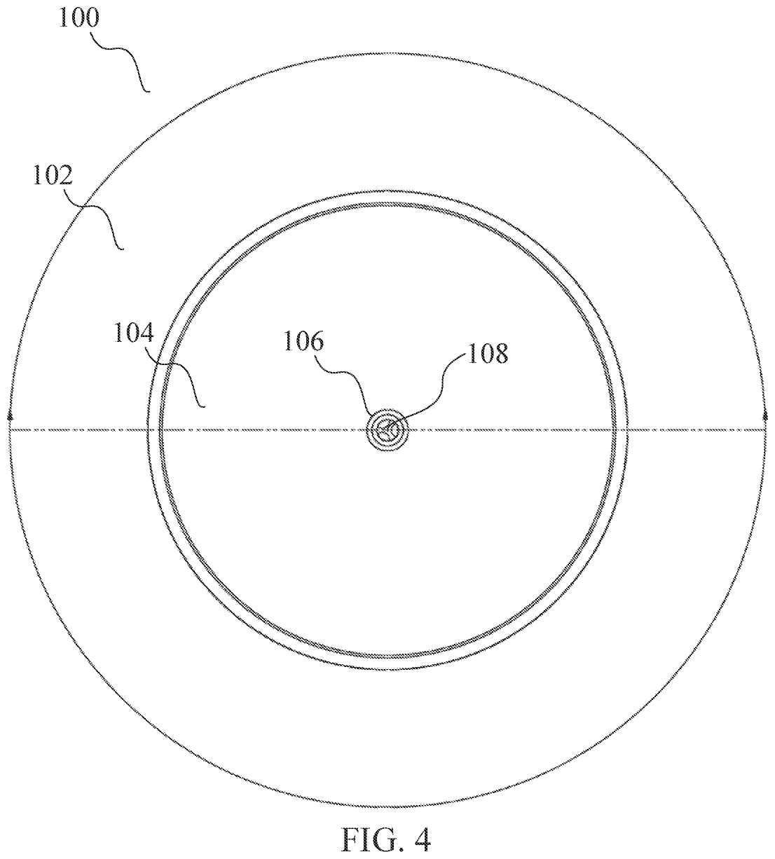

FIG. 4 is a top view of the biopotential detection device 100. As shown, shape of the housing 102 where it meets the skin area may be circular, as shown in FIG. 3. In other embodiments, the shape of the housing 102 where it meets the skin area may be oblong, or square.

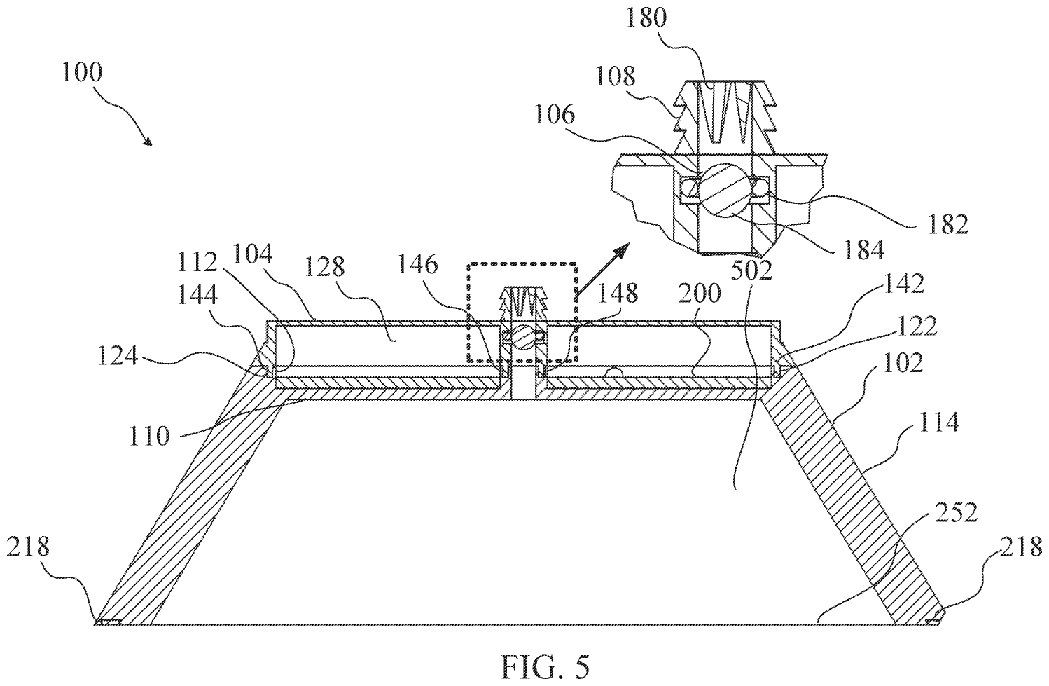

FIG. 5 is a cross-sectional view of the biopotential detection device 100, according to an embodiment. The bottom edge 252 of the skirt 114 may be configured to engage the skin area to form a vacuum chamber 502 defined by the skin area, the skirt 114, and the end wall 110 of the housing 102. In an example embodiment, the vacuum chamber 502 of the housing 102 may be the mechanism for creating a vacuum to secure the housing 102 to the skin area.

In some embodiments, the cover 104 may couple to the housing 102 by an engagement feature. For example and as shown in FIG. 5, the cover 104 includes a first axial flange 142 and a second axial flange 144. The first axial flange 142 is structured to fit within the first circumferential channel 122 and the second axial flange 144 is structured to fit within the second circumferential channel 124. The cover 104 may also include a third axial flange 146 that fits within a circular channel 148 of the raised central portion 126. The first axial flange 142, the second axial flange 144, and the third axial flange 146 are structured to couple the cover 104 to the housing 102. In additional and/or alternative embodiments, the cover 104 may couple to the housing 102 by a different engagement feature, such as a threaded engagement, a snap-fit engagement, a magnetic engagement, etc. In yet other embodiments, the cover 104 may be unitarily formed with the housing 102. All such variations are intended to fall within the scope of the disclosure.

The vacuum port 106 of the biopotential detection device 100 may extend into the vacuum chamber 502. In some embodiments, the vacuum port 106 may be configured to be connected to a pump or other suitable device at hose fitting 108. The pump may remove air from the vacuum chamber 502 of the housing 102. The pump may include at least one of a motorized vacuum pump, a handheld pump, a syringe, and/or other suitable device. By vacuuming air out of the vacuum chamber 502, a fluid-tight seal may be created between the bottom edge 252 of the skirt 114 and the skin area. The fluid-tight seal may improve the contact between the biopotential detection device 100 and the skin area.

As shown in FIG. 5, the gasket 182 and the ball gasket 184 are positioned within the vacuum port 106. The when the ball gasket 184 meets the gasket 182 it creates a fluid-tight seal in the vacuum port 106. The pronged member 180 is positioned at an upper end of the vacuum port 106 (e.g., proximal the hose fitting 108) such that the ball gasket 184 is retained by the pronged member 180 when air is being drawn out of the vacuum chamber 502 via the vacuum port 106.

In an example operating scenario, air is drawn out of the vacuum chamber 502 through the vacuum port 106. As briefly described above, the partial vacuum created within the vacuum chamber 502 results in a lower pressure within the vacuum chamber 502, enabling atmospheric air pressure to hold the biopotential detection device 100 to the skin area by suction. The ball gasket 184, the gasket 182, and pronged member 180 may allow for a unidirectional flow of air from the vacuum chamber 502 to an exterior of the housing 102. When the air is being drawn out of the vacuum chamber 502, the ball gasket 184 is retained by the pronged member 180. The pronged member 180 includes one or more gaps for air to move through the vacuum port 106. When the air has been drawn out of the vacuum chamber 502, the ball gasket 184 is forced into the gasket 182 forming a fluid-tight seal therebetween such that air is substantially prevented from entering the vacuum chamber 502. In other embodiments, a different form of valve may be used to allow for unidirectional flow of air from the vacuum chamber 502 to an exterior of the housing.

The biopotential detection device 100 also includes one or more electrodes 218. As shown in FIG. 5, in some embodiments, the biopotential detection device 100 includes two electrodes 218 with ends disposed at the bottom edge 252. As shown, the electrodes 218 are disposed diametrically opposite each other.

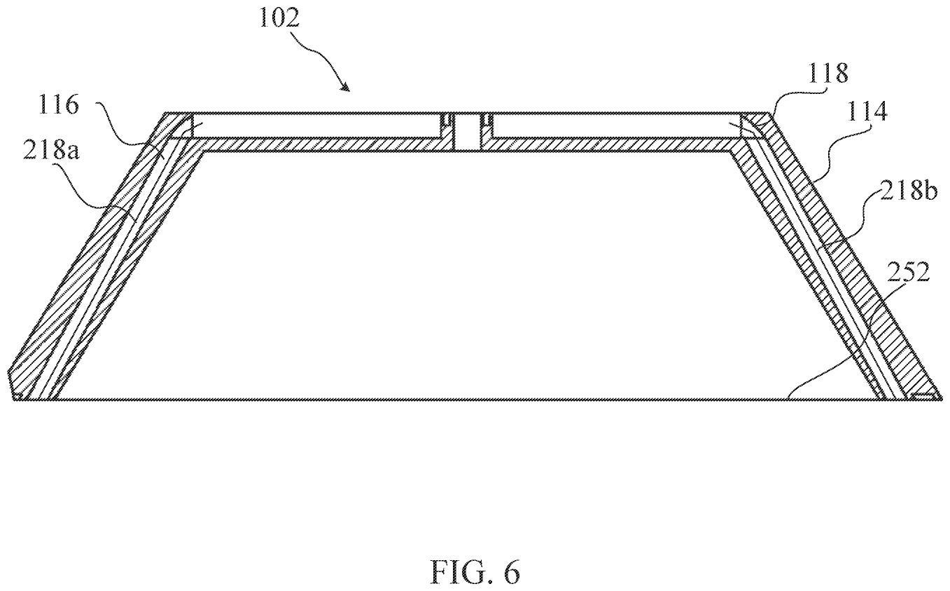

FIG. 6 is a cross section view of the housing 102 of the biopotential detection device 100 of FIG. 1. As shown, the first channel 116 and the second channel 118 extend through the skirt 114 and open at the bottom edge 252 of the housing 102. The biopotential detection device 100 includes one or more electrodes shown as a first electrode 218a and a second electrode 218b. The first electrode 218a extends from the end wall 110, through the first channel 116, and out the housing 102. The second electrode 218b extends from the end wall 110, through the second channel 118, and out of the housing 102. A first end of the electrodes 218 may be communicatively and/or electrically coupled to the controller 202. A second end of the electrodes 218 may be positioned on the bottom edge 252 of the skirt 114 such that, when the biopotential detection device 100 is placed on the skin area, and a vacuum is formed in the vacuum chamber 502, the electrodes 218a, 218b abut the skin area. When air is vacuumed out of the vacuum chamber 502, the electrodes 218a, 218b may be pushed into the skin area. When the electrodes 218a, 218b pushed into the skin area, the contact between the electrodes 218a, 218b and the skin may improve resulting in more reliable readings. In one embodiment, the electrodes 218a, 218b may be made of a metal, metal alloy, or other suitable material such as titanium nitrate or silver chloride.

The electrodes 218a, 218b are electrically and/or communicatively coupled to the controller 202. The controller 202 is positioned in the cavity 128. The controller 202 may further detect the biopotential via the at least two electrodes 218a, 218b. As shown, the first channel 116 and the second channel 118 are arranged opposite each other on the diameter of the housing 102. In an example embodiment, the ends of the electrodes 218a, 218b are no more than 2.5 inches apart (e.g., along the diameter of the bottom edge 252. In alternative embodiments, the first channel 116 and the second channel 118 are closer to each other on one side of the skirt 114 than the other, such that they are not diametrically opposed.

In an example embodiment, a bottom end of the first electrode 218a and a bottom end of the second electrode 218b may be disposed opposite each other on the bottom edge 252. The one or more electrodes 218a, 218b may be spaced apart by a predetermined distance. In some embodiments, the predetermined distance may be approximately 1.5 inches (e.g., within 10% of 1.5 inches). In other embodiments, the predetermined distance may be between 1 inch and 2.5 inches. In yet other embodiments, the predetermined distance may be less than 1 inch or greater than 2.5 inches.

In one embodiment of the biopotential detection device 100, the housing 102 has a width. The width may be the distance between opposing sides of the skirt 114. Alternatively, the width may include the depth of each side of the skirt 114. In some embodiments the width of the housing 102 is no more than 2.5 inches across. The housing 102 may also be no less than 1 inch wide.

The housing 102 of the biopotential detection device 100 may be formed in various manners. In an example embodiment, the housing 102 may be 3D printed. Alternatively, the housing 102 may be formed by stamping, extruding, or casting. The housing 102 may also be formed from various materials. For example, the housing 102 may be formed from one or more of polylactic acid, polycaprolactone, high density polyethylene, polypropylene, acrylonitrile butadiene tyrene, and/or any other suitable material.

The controller 202 may be structured to detect, by the electrodes 218, 218a, 218b, biopotential data. The biopotential data may include one or more biopotential signals, such as a voltage, a change in voltage, an average voltage, etc. the controller 202 is communicatively coupled to a remote computing device such that the controller 202 may send the data to be processed or analyzed at the remote computing device. Accordingly, the controller 202 may include a communication interface having a wireless transmitter. The controller 202 may be further configured to cause the wireless transmitter to transmit data the biopotential data to the remote computing device. The wireless transmitter may be one of a Wi-Fi transmitter, a Bluetooth transmitter, an NFC transmitter, and the like.

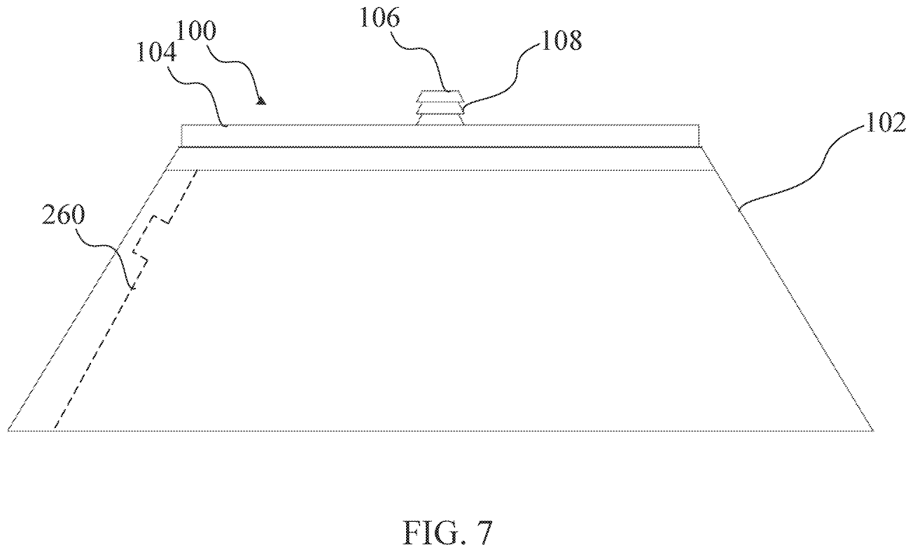

Now referring to FIG. 7, a side view showing aspects of the biopotential detection device 100 is shown, according to an example embodiment. The biopotential detection device 100 is shown to include a puncture surface 260. The puncture surface 260 may extend around at least a portion of the skirt 114. The puncture surface 260 defines a portion of the skirt 114. In some embodiments, the puncture surface 260 has a thinner wall thickness. For example, the skirt 114 may have a first portion with a first wall thickness and a second portion, defined by the puncture surface 260, with a second wall thickness, wherein the second wall thickness is thinner than the first wall thickness. The puncture surface 260 is puncturable to allow air to flow into the vacuum chamber 502 thereby releasing the vacuum to the skin area.

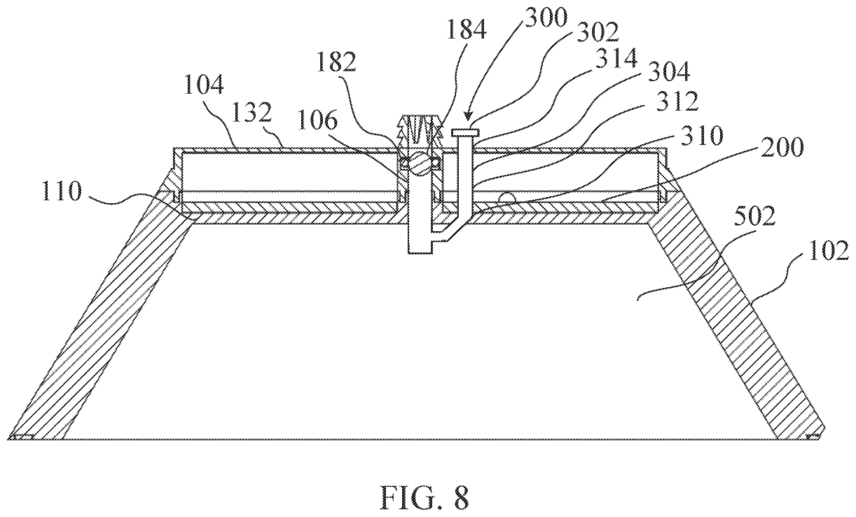

FIG. 8 is a cross-sectional view showing aspects of the biopotential detection device of FIG. 1. The biopotential detection device 100 is shown to include a valve assembly 300. The valve assembly includes a valve 302 and a tube 304. The valve 302 is operable between an open positon and a closed position. In the open positon a fluid is allowed to flow through the valve 302 and the tube 304. In the closed position, a fluid is substantially prevented from flowing through the valve 302 and the tube 304. A first end of the tube 304 is in fluid communication with the vacuum port 106 and a second end of the tube is disposed at the valve 302. The tube 304 may extend through the end wall 110, the control circuit 200 and the top surface 132. Accordingly, the end wall 110 includes a second housing opening 310, the control circuit 200 includes a second control opening 312, and the top surface 132 includes a second cover opening 314. The second housing opening, the second control opening 312, and the second cover opening 314 define a space through which the tube 304 extends through.

In some embodiments, the valve assembly 300 may be provided in addition to the gasket 182, ball gasket 184, and pronged member 180. In other embodiments, the valve assembly 300 may be provided instead of the gasket 182, ball gasket 184, and pronged member 180. In some embodiments, the valve assembly 300 may be provided in addition to and/or instead of the puncture surface 260.

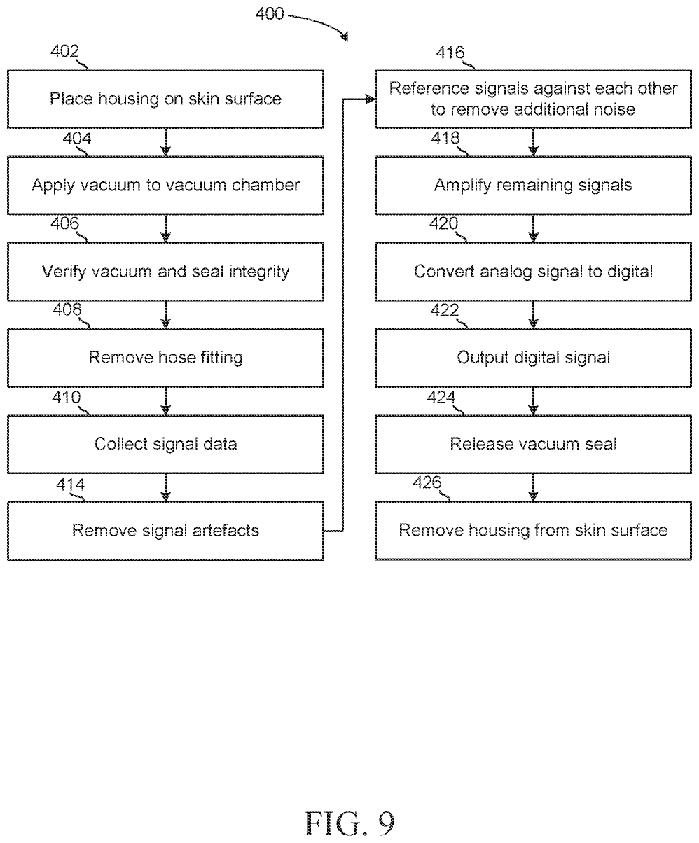

FIG. 9 is a flow diagram of a method 400 of operating the biopotential detection device 100 of FIG. 1, according to an example arrangement. In broad overview of method 400, at step 402, the housing is placed on a skin surface. At step 404, a vacuum is applied to the vacuum chamber. At step 406, the vacuum and seal integrity is verified. At step 408, the hose fitting is removed. At step 410, the biopotential detection device 100 collects signal data. At step 414, the controller 202 removes signal artefacts. At step 416, the controller 202 references signals against each other to remove additional noise. At step 418, the controller 202 amplifies the remaining signals. At step 420, the controller converts the analog signals to a digital signal. At step 422, the controller 202 outputs the digital signals. At step 424, the vacuum seal is released. At step 426, the housing is removed from the skin surface. In some arrangements, the steps of the method 400 may be performed in a different order than as shown in FIG. 9. For example, step 408 may be performed after step 422. In some arrangements, the method 400 may include more or fewer steps than as shown in FIG. 9. For example, step 406 and/or step 408 may be excluded from the method 400.

Referring to the method 400 in more detail, at step 402, the housing 102 is placed on a skin surface. In some embodiments, when the housing 102 includes the adhesive ring 250, the adhesive ring 250 temporarily adheres to the skin surface.

At step 404, a vacuum is applied to the vacuum chamber. As described above, a hose of a vacuum device may be coupled to the biopotential detection device 100 at the hose fitting 108. The vacuum device may draw air out of the vacuum chamber 502. As briefly described above, the hose may be any suitable tube for applying a vacuum to the vacuum chamber 502. In an example embodiment, the hose may have an inner diameter between 1/32 inch and ½ inch. In another example embodiment, the hose may have an inner diameter is between ⅛ inch and 3/16 inch. As described above, the vacuum device may be any suitable vacuum device including a handheld device, a syringe, an electric or motorized vacuum, and so on. At step 406, the vacuum and seal integrity is verified. For example, a user may verify that no air is leaking into the vacuum chamber 502 past the gasket 182 and ball gasket 184. In another example, a user may apply upward (e.g., away from the skin surface) pressure to the housing 102 to verify that the housing is attached to the skin surface. In embodiments where the bottom edge 252 defines a sealing surface, the sealing surface may form a circumferential seal around the skirt 114. A user may visually inspect the sealing surface to verify that the sealing surface has formed a seal around the circumference of the skirt 114.

At step 408, the hose fitting 108 is removed. In some embodiments, the hose fitting 108 is removably coupled to the cover 104 such that a user may remove the hose fitting 108 and replace the hose fitting 108 as needed. In some embodiments, the hose fitting 108 is fixed to the cover 104. A user may, after step 406, break the hose fitting 108 from the cover 104. In these embodiments, the hose fitting 108 cannot be easily re-attached to the cover 104.

At step 410, the biopotential detection device 100 collects signal data. For example, the controller 202 may detect, by the one or more electrodes 218, 218a, 218b, one or more biopotential signals. At step 414, the controller 202 removes signal artefacts. The artefacts may be caused by one or more background devices. The controller 202 may automatically correct for standard background devices. For example, the controller 202 may automatically correct for a light source operating at 60 Hz. At step 416, the controller 202 references signals against each other to remove additional noise. For example, the controller 202 may analyze one or more signals from one or more electrodes 218, 218a, 218b and to remove any additional noise. At step 418, the controller 202 amplifies the remaining signals. At step 420, the controller converts the analog signals to a digital signal.

At step 422, the controller 202 outputs the digital signals. For example, the controller 202 may output the digital signals by a wireless communication device such as a Bluetooth device, a NFC device, a Wi-Fi device, and so on.

At step 424, the vacuum seal is released. In some embodiments, the vacuum seal may be released by puncturing the puncture surface 260. In other embodiments, the vacuum seal may be released by opening a valve 302. At step 426, the housing is removed from the skin surface.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using one or more separate intervening members, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic. For example, circuit A communicably “coupled” to circuit B may signify that the circuit A communicates directly with circuit B (i.e., no intermediary) or communicates indirectly with circuit B (e.g., through one or more intermediaries).

While various circuits with particular functionality are described herein, it should be understood that the controller 202 may include any number of circuits for completing the functions described herein. For example, the activities may be distributed into multiple circuits or combined as a single circuit. Additional circuits with additional functionality may also be included. Further, the controller 202 may further control other activity beyond the scope of the present disclosure.

As mentioned above and in one configuration, the “circuits” (e.g., control circuit 200) may be implemented in machine-readable medium storing instructions (e.g., embodied as executable code) for execution by various types of processors, such as a processor. Executable code may, for instance, comprise one or more physical or logical blocks of computer instructions, which may, for instance, be organized as an object, procedure, or function. Nevertheless, the executables need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, biopotential data may be identified and illustrated herein within circuits, and may be embodied in any suitable form and organized within any suitable type of data structure. The biopotential data may be collected as a single data set, or may be distributed over different locations including over different storage devices, and may exist, at least partially, merely as electronic signals on a system or network.

While the term “processor” is briefly defined above, the term “processor” and “processing circuit” are meant to be broadly interpreted. In this regard and as mentioned above, the “processor” may be implemented as one or more processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations.

Embodiments within the scope of the present disclosure include program products comprising computer or machine-readable media for carrying or having computer or machine-executable instructions or data structures stored thereon. Such machine-readable media can be any available media that can be accessed by a computer. The computer readable medium may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. More specific examples of the computer readable medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device. Machine-executable instructions include, for example, instructions and data which cause a computer or processing machine to perform a certain function or group of functions.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. Computer readable program code embodied on a computer readable signal medium may be transmitted using any appropriate medium, including but not limited to wireless, wireline, optical fiber cable, Radio Frequency (RF), or the like, or any suitable combination of the foregoing

In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present disclosure may be written in any combination of one or more other programming languages, including an object oriented programming language such as Java, Smalltalk, C++ or the like and conventional procedural programming languages, such as the “C” programming language or similar programming languages. The computer readable program code may execute entirely on the user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

Although the Figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the apparatus and system as shown in the various exemplary embodiments is illustrative only. Additionally, any element disclosed in one embodiment may be incorporated or utilized with any other embodiment disclosed herein.

What is claimed is:

1. A biopotential detection device comprising:
a housing comprising:
an end wall; and
a skirt extending away the end wall in a first direction, the skirt having a bottom edge disposed away from the end wall and configured to engage a surface, wherein a void defined by the skirt, the end wall, and the skin area forms a vacuum chamber;
a cover disposed adjacent to the end wall, opposite the skirt;
a vacuum port extending through the cover and the end wall and into the vacuum chamber; and
one or more electrodes disposed on the bottom edge of the skirt.

2. The biopotential detection device of claim 1, wherein a cavity is defined at least in part by the cover and the end wall of the housing.

3. The biopotential detection device of claim 2, further comprising a control circuit disposed within the cavity, the control circuit comprising a controller structured to receive biopotential data from the one or more electrodes.

4. The biopotential detection device of claim 3, wherein the control circuit further comprises a wireless transmitter communicatively coupled to the controller, wherein the controller is further configured to cause the wireless transmitter to transmit the biopotential data.

5. The biopotential detection device of claim 1, wherein the skirt comprises a first portion having a first wall thickness and a second portion having a second wall thickness less than the first wall thickness; and
wherein second portion defines a puncture surface, wherein the puncture surface is puncturable such that air is allowed to flow into the vacuum chamber, thereby releasing the vacuum to the skin area when the puncture surface is punctured.

6. The biopotential detection device of claim 1, further comprising an adhesive ring disposed on at least a portion of the bottom edge of the wall.

7. The biopotential detection device of claim 1, further comprising a ball gasket, a circular gasket, and a pronged member disposed within the vacuum port;
wherein the pronged member is configured to temporarily retain the ball gasket while air is being drawn out of the vacuum chamber; and
wherein the ball gasket is forced into the circular gasket when the vacuum chamber is substantially free of air, such that the ball gasket, the circular gasket, and the pronged member allow for a unidirectional flow of air from the vacuum chamber to an exterior of the housing.

8. The biopotential detection device of claim 1, further comprising a valve assembly comprising a valve and a tube fluidly coupled to the valve and the vacuum port, the valve assembly configured to allow for a unidirectional flow of air from the vacuum chamber to an exterior of the housing.

9. The biopotential detection device of claim 1, further comprising a hose fitting engaged with the vacuum port.

10. The biopotential detection device of claim 1, further comprising:
a first channel in the skirt extending from the lip to the bottom edge of the skirt on a first radial side of the housing; and
a second channel in the skirt extending through the lip to the bottom edge of the skirt on a second radial side of the housing, opposite the first radial side;
wherein a first electrode of the one or more electrodes extends through the first channel.

11. The biopotential detection device of claim 1, wherein the cover comprises a first axial flange; and
wherein the lip comprises a first circumferential channel configured to receive the first axial flange such that the cover is coupled to the housing.

12. A housing comprising:
an end wall;
a lip extending around a circumference of the top portion and away from the top portion in a first direction;
a skirt extending away from the end wall in a second direction, oblique from the first direction, the skirt at least partially defining a vacuum chamber; and
a raised central portion having a housing opening extending there through, the housing opening defining a fluid pathway.

13. The housing of claim 12, wherein the raised central portion, the end wall, and the lip at least partially define a cavity structured to receive a controller.

14. The housing of claim 12, further comprising:
a first channel in the skirt extending through the lip to the bottom edge of the skirt on a first radial side of the housing; and
a second channel in the skirt extending through the lip to the bottom edge of the skirt on a second radial side of the housing, opposite the first radial side.

15. The housing of claim 12, wherein the lip further comprises a first circumferential channel extending at least partially between the first channel and the second channel on a third radial side of the housing.

16. A method comprising:
positioning a biopotential detection device on a skin area, wherein the biopotential detection device comprises a housing having a bottom edge that contacts the skin area and a vacuum chamber defined at least in part by the skin area and the housing;
securing the housing with at least two electrodes in contact with the skin area; and
suctioning air out of the vacuum chamber to as to create a vacuum seal between the housing and the skin area to secure the electrodes on the skin area.

17. The method of claim 16, wherein the securing comprises securing the housing to the skin by an adhesive ring disposed on at least a portion of the bottom edge of the housing.

18. The method of claim 16, wherein the suctioning comprises suctioning the air out of the vacuum chamber through a vacuum port extending from the vacuum chamber to an end wall of the housing;
wherein the vacuum port includes a hose fitting and the suctioning further comprises suctioning the air out of the chamber by a hose secured on the hose fitting.

19. The method of claim 16, further comprising:
puncturing through the housing into the vacuum chamber so as to release the vacuum seal between the housing and the skin.

20. The method of claim 16, further comprising
opening a valve of a valve assembly, the valve assembly defining a fluid path at least in part by the vacuum chamber and an exterior of the housing, such that the vacuum seal between the housing and the skin is released.
